(19) United States
(12) Patent Application Publication
(10) Pub. No.: US 2006/0184192 A1
(43) Pub. Date: Aug. 17, 2006

(54) Systems and methods for providing cavities in interior body regions

(76) Inventors: Aaron D. Markworth, Saddle Brook, NJ (US); Bryce Anton Way, San Jose, CA (US)

Correspondence Address: KILPATRICK STOCKTON LLP, 1001 WEST FOURTH STREET, WINSTON-SALEM, NC, 27101, US

(21) Appl. No.: 11/056,785

(22) Filed: Feb. 11, 2005

(51) Int. Cl. A61M 29/00

(52) U.S. Cl. 606/198

(57) ABSTRACT

Systems and methods for providing a cavity in an interior body region are described. In one described method, a shaft comprising an expansible portion configured to expand once beyond a distal end of a first cannula is inserted through the first cannula. The expansible portion is then expanded, thereby creating a cavity. While the expansible portion is expanded, a bone cement may be inserted into the cavity. The expansible portion may then be collapsed, and the shaft removed through the first cannula.

### FIELD OF THE INVENTION

The invention relates to systems and methods for providing cavities in interior body regions for diagnostic or therapeutic purposes.

### BACKGROUND

Certain diagnostic or therapeutic procedures require provision of a cavity in an interior body region. For example, as disclosed in U.S. Pat. Nos. 4,969,888 and 5,108,404, an expansible body may be deployed to form a cavity in cancellous bone tissue, as part of a therapeutic procedure that fixes fractures or other abnormal bone conditions, both osteoporotic and non-osteoporotic in origin. The expansible body may compress the cancellous bone to form an interior cavity. The cavity may receive a filling material, such as a bone cement, which provides renewed interior structural support for cortical bone.

This procedure can be used to treat cortical bone, which due to osteoporosis, avascular necrosis, cancer, trauma, or other disease is fractured or is prone to compression fracture or collapse. These conditions, if not successfully treated, can result in deformities, chronic complications, and an overall adverse impact upon the quality of life.

A demand exists for further systems and methods that are capable of providing cavities in bone and other interior body regions in safe and efficacious ways.

### SUMMARY

Embodiments of the present invention provide systems and methods for providing cavities in interior body regions. One illustrative embodiment comprises inserting through a first cannula a shaft comprising an expansible portion configured to expand once beyond a distal end of the first cannula. This illustrative embodiment further comprises expanding the expansible portion, thereby creating a cavity, and inserting a bone cement into the cavity while the expansible portion is expanded. The expansible portion may then be collapsed, and the shaft removed through the first cannula. The bone cement may remain to support a surrounding structure, such as a vertebral body.

This embodiment is mentioned not to limit or define the invention, but to provide an example of an embodiment of the invention to aid understanding thereof. Illustrative embodiments are discussed in the Detailed Description, and further description of the invention is provided there. Advantages offered by the various embodiments of the present invention may be further understood by examining this specification.

### BRIEF DESCRIPTION OF THE FIGURES

These and other features, aspects, and advantages of the present invention are better understood when the following Detailed Description is read with reference to the accompanying drawings, wherein:

FIG. 1 is a perspective view of a tool according to one embodiment of the present invention;

FIG. 2 is an enlarged end view of the tool shown in FIG. 1, wherein the expansible portion thereof is shown in an unexpanded state;

FIG. 3 is an enlarged end view of the tool shown in FIG. 1, wherein the expansible portion thereof is shown in an expanded state;

FIG. 4 is an elevation view of the tool shown in FIG. 1, wherein the expansible portion thereof is shown in a retracted position;

FIG. 5 is a perspective view of the tool shown in FIG. 1, wherein the expansible portion thereof is shown in an expanded state;

FIG. 6 is an elevation view of the tool shown in FIG. 1, wherein the expansible portion thereof is shown in a partially retracted position;

FIG. 7 is a perspective view of a balloon-actuated expansible portion of a tool according to one embodiment of the present invention;

FIG. 8 is an elevation (lateral) view of several human vertebrae, with a cannula establishing a percutaneous path to a vertebral body of one of the several vertebrae;

FIG. 9 is a plan (coronal) view of a human vertebra being accessed by a cannula, with portions removed to reveal cancellous bone within the vertebral body;

FIG. 10 is an elevation (lateral) view of a human vertebra comprising a vertical compression fracture condition and with a tool comprising a balloon-inflatable expansible portion shown in an unexpanded state according to one embodiment of the present invention deployed to provide a cavity within the vertebra;

FIG. 11 is an elevation view of the tool and vertebra of FIG. 10, wherein the expansible portion of the tool is shown in an expanded state, and the vertebra is shown with an increased internal dimension resulting from the expansion of the expansible portion;

FIG. 12 is a plan (coronal) view of the tool and vertebra of FIG. 10, wherein the tool is shown being rotated in cancellous bone;

FIG. 13 is a flow chart of a method according to one embodiment of the present invention;

FIG. 14 is a plan view of a sterile kit configured to store a single use tool according to one embodiment of the present invention; and

FIG. 15 is an exploded perspective view of the sterile kit of FIG. 14.

### DETAILED DESCRIPTION

Embodiments of the present invention provide systems and methods for providing cavities in interior body regions. The systems and methods embodying the invention can be adapted for use in many suitable interior body regions, wherever the formation of a cavity within or adjacent one or more layers of tissue may be required for a therapeutic or diagnostic purpose. The illustrative embodiments show the invention in association with systems and methods used to treat bones. In other embodiments, the present invention may be used in other interior body regions or types of tissues.

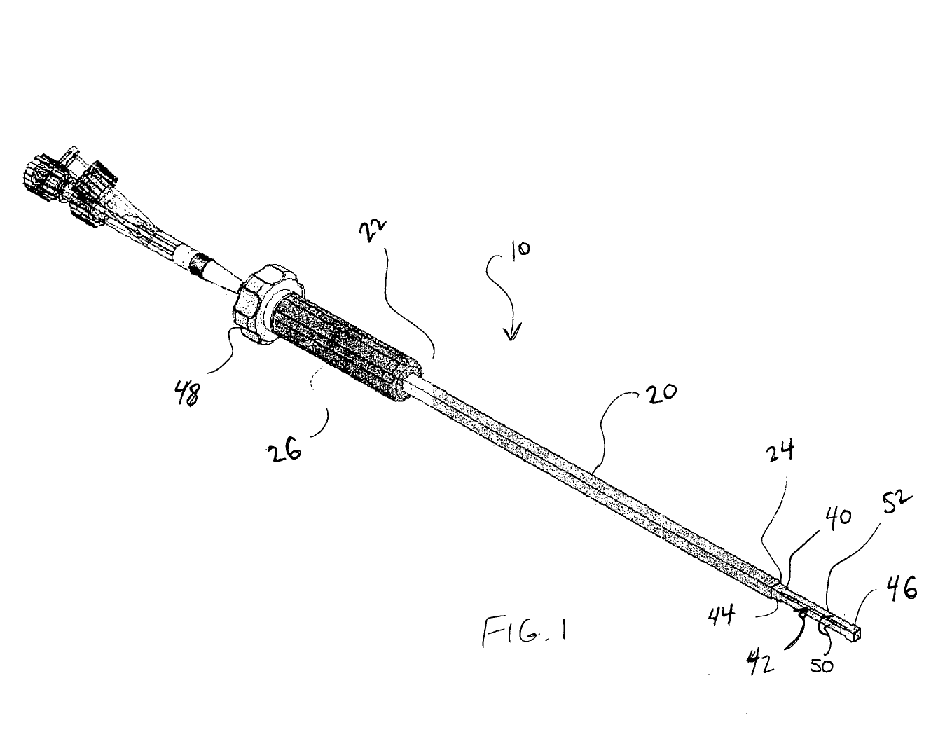

Referring now to the Figures, in which like part numbers depict like elements throughout the Figures, FIG. 1 is a perspective view of a tool 10 according to one embodiment of the present invention. The tool 10 shown in FIG. 1 comprises a surgical instrument, and is configured to allow an operator to provide a cavity in a targeted treatment area. The tool 10 comprises an outer shaft 20 having a proximal end 22 and a distal end 24. The outer shaft 20 may be made from a resilient inert material providing torsion transmission capabilities (e.g., stainless steel, a nickel-titanium alloy such as Nitinol, and other suitable metal alloys). The outer shaft 20 may comprise a handle 26 to aid in gripping and maneuvering the outer shaft 20. For example, in one embodiment, the handle 26 can be made of a foam material secured about the outer shaft 20.

The tool 10 shown in FIG. 1 further comprises an inner shaft 40 configured to move with respect to the outer shaft 20. The inner shaft 40 may be made from a resilient inert material providing torsion transmission capabilities (e.g., stainless steel, a nickel-titanium alloy such as Nitinol, and other suitable metal alloys). In other embodiments, the inner shaft 40 may be fashioned from a variety of suitable materials, comprising a carbon fiber, a glass, or a flexible material, such as a plastic or rubber. In one embodiment comprising a flexible inner shaft 40, the inner shaft 40 may be, for example, fashioned from twisted wire filaments, such stainless steel, nickel-titanium alloys (such as Nitinol), and suitable other metal alloys.

In the embodiment shown in FIG. 1, both the outer shaft 20 and the inner shaft 40 are fabricated from square tubing, comprise a substantially square cross-section, and a bore therethrough along a common axis. In other embodiments one or both of the outer shaft 20 and the inner shaft 40 can, in cross section, be round, rectilinear, elliptical, polygonal, or any other suitable configuration. In another embodiment, at least a portion of the inner shaft 40 may be solid.

The inner shaft 40 comprises an expansible portion 42 disposed between a proximal portion 44 and a distal portion 46. The expansible portion 42 is constrained from expanding beyond an expanded dimension by the proximal portion 44 and the distal portion 46. In the embodiment shown, the cross section of the proximal portion 44 is substantially similar to the cross section of the distal portion 46, whether the expansible portion 42 is in an expanded state, or in an unexpanded state.

In the embodiment shown, the expansible portion 42 comprises a plurality of outwardly expanding beams 50. In the embodiment shown in FIGS. 1-6, the plurality of beams 50 are provided by removing material from each of the four corners of the inner shaft 40 along the expansible portion 42. In other embodiments, the expansible portion 42 may be provided using another suitable method. For example, the expansible portion 42 may be provided by stretching a portion of the inner shaft 40, or by providing one or more slits, grooves, depressions, or holes in a portion of the inner shaft 40 located between the proximal portion 44 and the distal portion 46.

In the embodiment shown in FIG. 1, the material from which the plurality of beams 50 are fashioned is the same as that of the inner shaft 40. In other embodiments, at least the expansible portion 42 of the inner shaft 40 may be fashioned from a material different than another portion of the inner shaft 40. For example, in one embodiment, the expansible portion 42 may be fashioned from a surgical grade shape memory material formed with a resilient memory, such as a nickel titanium alloy, and the proximal portion 44 of the inner shaft 40 may be fashioned from surgical grade stainless steel, a plastic, or any other suitable material.

The plurality of beams 50 shown in FIG. 1 each comprise a trapezoidal cross section. In other embodiments, one or more of the beams 50 can, in cross section, be round, rectilinear, or any other suitable configuration.

In the embodiment shown in FIG. 1, each of the plurality of beams 50 of the expansible portion 42 comprises at least one beveled edge 52 formed by removal of material at a suitable angle from one or more of the corners of the square tubing of the inner shaft 40. In other embodiments, none of the beams 50 or fewer than all of the beams 50 may comprise one or more beveled edges 52. In yet another embodiment, another portion of the expansible portion 42 may comprise a surface configured to directly contact and shear at least one layer of tissue.

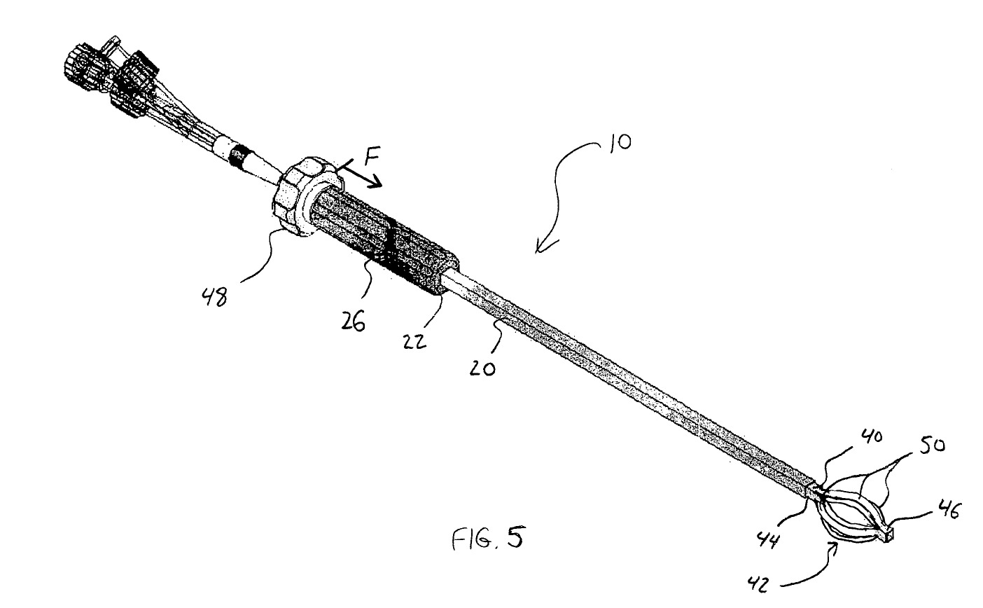

When the expansible portion 42 is at least partially expanded, as shown in FIGS. 3, 5, and 6, at least one of the beveled edges 52 of one or more of the plurality of beams 50 may be used to directly contact and shear (curette) at least one layer of body tissue adjacent the expansible portion 42 as the inner shaft 40 is rotated. In the embodiment shown, the expansible portion 42 is configured to cut adjacent tissue mass in the targeted treatment area when rotated. In another embodiment, the proximal end of at least one of the outer shaft 20 and the inner shaft 40 may carry a fitting (not shown) that, in use, may be coupled to an electric motor (not shown). The motor may thus rotate one or both of the inner shaft 40 and the outer shaft 20, thereby rotating the expansible portion 42.

In the embodiment shown in FIGS. 1-6, the inner shaft 40 comprises one continuous piece of material from which plurality of beams 50 have been provided by removing some the continuous material. In other embodiments, the expansible portion 42 may be coupled to at least one of the proximal portion 44 and the distal portion 46 via another suitable method. For example, in one such embodiment, a expansible beam 50 of an expansible portion 42 may be coupled to both the proximal portion 44 and the distal portion 46 through the use of welding, gluing, melting, or any other suitable fastener (such as a screw, a rivet, a tack, a staple, a nail, etc.).

The expansible portion 42 of the inner shaft 40 shown in FIGS. 1-6 is in communication with a controller 48. The controller 48 shown in FIG. 1, and FIGS. 4-6 comprises a slide controller. In other embodiments, at least the expansible portion 42 of the inner shaft 40 may be in communication with a different suitable type of controller, such as a pistol grip controller, a ratcheting controller, a threaded controller, or any other suitable type of controller that can be configured to permit an operator of the tool 10 to control at least one of the extent to which the expansible portion 42 extends beyond the distal end 24 of the outer shaft 20, and the extent to which the expansible portion 42 is expanded.

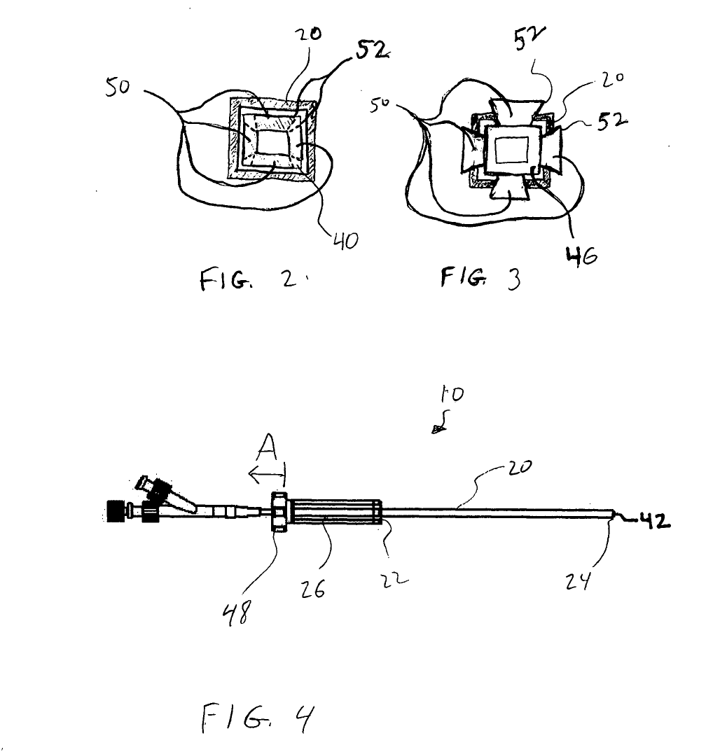

Referring now to FIG. 2, an enlarged end view of the tool 10 shown in FIG. 1 is shown, wherein the expansible portion 42 is shown in an unexpanded state. As shown in FIG. 2, while the expansible portion 42 of the inner shaft 40 is in the unexpanded state, an unexpanded dimension of the expansible portion 42, as measured perpendicular to the axis of the inner shaft 40, comprises a lesser dimension than the smallest inside bore dimension of the hollow outer shaft 20, thereby providing a clearance between the inner shaft 40 and the outer shaft 20 that permits the inner shaft 40 to move within and along the axis of the outer shaft 20. In other embodiments, the inner shaft 40 may be configured to be rotated with respect to the outer shaft 20.

As shown in FIG. 2 and described above, the expansible beams 50 are formed by removing the four corners of the square tubing from which the inner shaft 40 is formed along the expansible portion 42. In the embodiment shown, each of the four corners of the square tubing inner shaft 40 have been removed at such an angle that the resulting expansible beams 50 comprise a trapezoidal cross sectional shape with each side of the trapezoid comprising a beveled edge 52. In other embodiments, one or more of the beams 50 may comprise a cross-section of another suitable shape, such as round, rectilinear, polygonal, asymmetrical, etc.

Referring now to FIG. 3, an enlarged end view of the tool 10 is shown, wherein the expansible portion 42 is shown in an expanded state. As shown in FIG. 3, when in the expanded state, the expansive portion 42 comprises an expanded dimension, as measured perpendicular to the axis of the inner shaft 40, that is at least as large as the largest inside dimension of the interior bore of the outer shaft 20. It can also be seen that while in this expanded state, the distal portion 46 remains substantially unchanged in dimension from the unexpanded state shown in FIG. 2. In the present embodiment, the expansible portion 42 is constrained from expanding beyond the expanded dimension shown in FIG. 3 by the unexpanded proximal portion 44, and the unexpanded distal portion 46.

In the embodiment shown in FIGS. 1-6, the beveled edges 52 of the expansible beams 50 provide a surface that may be used to directly contact and shear adjacent tissue when the expansible portion 42 is in the expanded state shown in FIG. 3, and at least the inner shaft 40 is rotated about its axis.

Referring now to FIG. 4, an elevation view of the tool 10 is shown, wherein expansible portion 42 is shown in a retracted position. As shown in FIG. 4, sliding the controller 48 in the proximal direction (arrow A in FIG. 4) shortens or eliminates the distance the expansible portion 42 extends from the distal end 24 of the outer shaft 20, thereby at least partially limiting expansion of the expansible portion 42. As shown in FIG. 4, in its most proximal position, the expansible portion 42 is withdrawn to a point within the distal end 24 of the outer shaft 20, thereby constraining the expansible beams 50 of the expansible portion 42 of the inner shaft 40 to the unexpanded state shown in FIG. 2.

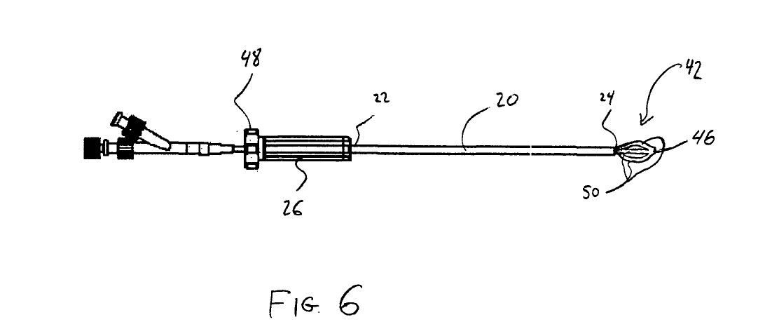

Referring now to FIG. 5, a perspective view of the tool 10 is shown, wherein the expansible portion 42 is shown in the expanded state. As shown in FIG. 5, sliding the controller 48 in the distal direction (arrow F in FIG. 5) advances the expansible portion 42 to a point beyond the distal end 24 of the outer shaft 20, exposing the beams 50 of the expansible portion 44 to adjacent body tissue. Once the expansible portion 42 of the inner shaft 40 is at least partially out of the distal end 24 of the outer shaft 20, the physician may expand the dimension of the expansible portion 42 from the unexpanded state (see FIG. 2) to the expanded state (see FIGS. 3 and 5), or to any intermediate state (see FIG. 6).

Referring now to FIG. 6, an elevation view of the tool 10 is shown, wherein the expansible portion 42 is shown partially retracted from a point beyond the distal end 24 of the outer shaft 20 (as shown in FIG. 5) to a point where at least part of the expansible portion 42 is within the distal end 24 of the outer shaft 20. As shown in FIG. 6, an operator of the tool 10 may be able to contract the expansible portion 42 from the expanded state shown in FIGS. 3 and 5 back to the unexpanded state shown in FIG. 2, or to any intermediate state by sliding the controller 48 in the proximal direction from its most distal position. As the controller 48 is slid proximally, the expansible portion 42 of the inner shaft 40 is at least partially returned to a point within the distal end 24 of the outer shaft 20 (as shown in FIG. 6). In the embodiment shown in FIGS. 1-6, pressure resulting from contact between the outer shaft 20 and the plurality of beams 50 causes the beams 50 of the expansible portion 42 to deflect toward a central axis of the inner shaft 40, thereby at least partially contracting the expansible portion 42 (for example, to the extent the expansible portion 42 is expanded in FIG. 5, as compared to the extent to which it is expanded in FIG. 6).

In one embodiment of the present invention, the controller 48 can also comprise indicia by which the physician can visually estimate the extent to which the expansible portion 42 is extended beyond the distal end 24 of the outer shaft 20, or the extent to which the expansible portion 42 has been expanded.

In another embodiment of the present invention, at least a portion of the expansible portion 42 may comprise one or more radiological markers. For example, in the embodiment shown in FIGS. 1-6, one or more of the plurality of beams 50 may comprise one or more radiological markers. The markers may be fashioned from a radiopaque material, such as platinum, gold, calcium, tantalum, and other heavy metals.

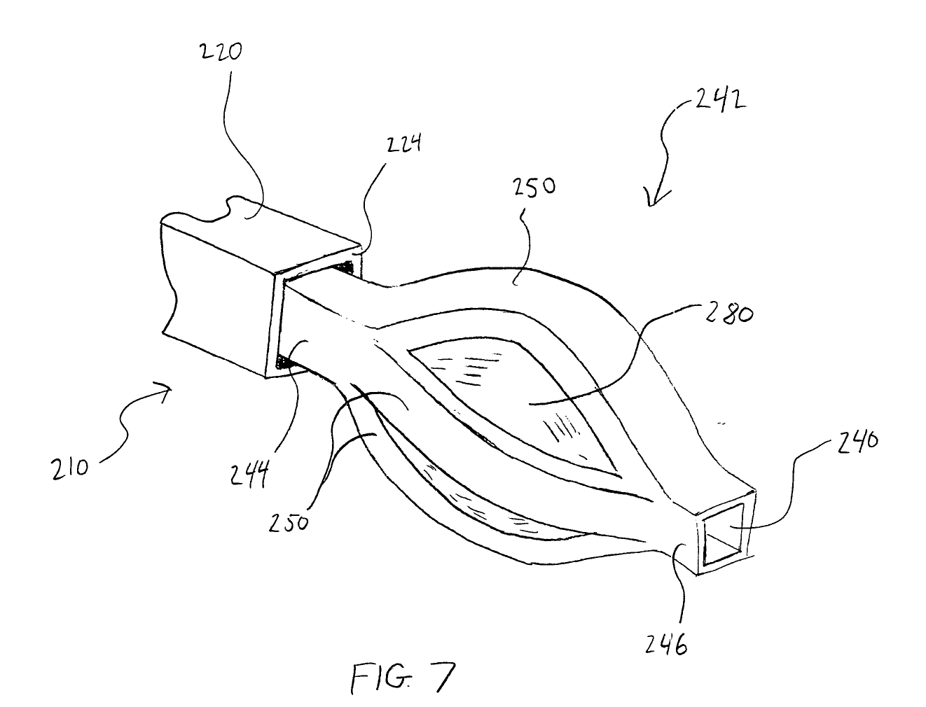

In an embodiment employing a plurality of radiological markers, a first marker may be placed at or near a point where the expansible portion 42 is coupled to the distal portion 46, while another marker may be placed at a location on the expansible portion 42 spaced apart from the first marker. In another embodiment, the distal portion 46 of the inner shaft 40, or the distal end 24 of the outer shaft 20 can carry one or more markers. A radiological marker may permit radiologic visualization of the expansible portion 42 and outer shaft 20 within a targeted treatment area. In other embodiments, other forms of markers can be used to allow the physician to visualize the location and shape of at least the expansible portion 42 within the targeted treatment area. For example, in one embodiment wherein the expansible portion 42 is expanded using a force provided by an inflatable balloon therewithin (see FIG. 7), the balloon may be inflated with a radiopaque material.

A tool according to one embodiment of the present invention, such as the tool 10 described with respect to FIGS. 1-6, can comprise an interior lumen. The lumen may be coupled via a Y-valve to an external source of fluid and an external vacuum source. In one such embodiment, a rinsing liquid, e.g., sterile saline, can be introduced from the source through the lumen into the targeted tissue region before, during or after the tool 10 provides a cavity in a tissue mass. The rinsing liquid may reduce friction and conduct heat away from the tissue during a cutting operation. The rinsing liquid can be introduced continuously or intermittently while the tissue mass is being cut. The rinsing liquid can also carry an anticoagulant or other anti-clotting agent. In one such embodiment, the lumen may be coupled to the vacuum source, and liquids and debris can be aspirated from the targeted tissue region through the lumen.

Referring now to FIG. 7, a perspective view of a balloon-actuated expansible portion 242 of a tool 210 according to one embodiment of the present invention is shown. As shown in FIG. 7, an expansible portion 242 of an inner shaft 240 has been extended beyond a distal end 224 of an outer shaft 220. An inflatable balloon 280 has been inserted into an interior bore extending through an inner shaft 240 along the axis of the outer shaft 220. The balloon 280 shown is inflated with a material (such as a radiopaque material as described above) through an aperture (not shown) facing a proximal portion 244 of the inner shaft 240.

In the embodiment shown, as the balloon 280 is inflated, pressure on the surface of the balloon 280 causes expansible portion 242 of the inner shaft 240 to expand. In the embodiment shown, the expansible portion 242 comprises a plurality of expansible beams 250, each coupled on both a proximal end and a distal end to the proximal and distal portions 244, 246, respectively, of the inner shaft 240.

In the embodiment shown in FIG. 7, the balloon 280 may be deflated and removed from the inner shaft 240 once the expansible portion 242 has been expanded. At least the expansible portion 242 of the inner shaft may be fashioned from a material capable of substantially holding the expansible portion 242 in the expanded state shown in FIG. 7 after the balloon 280 has been deflated and removed.

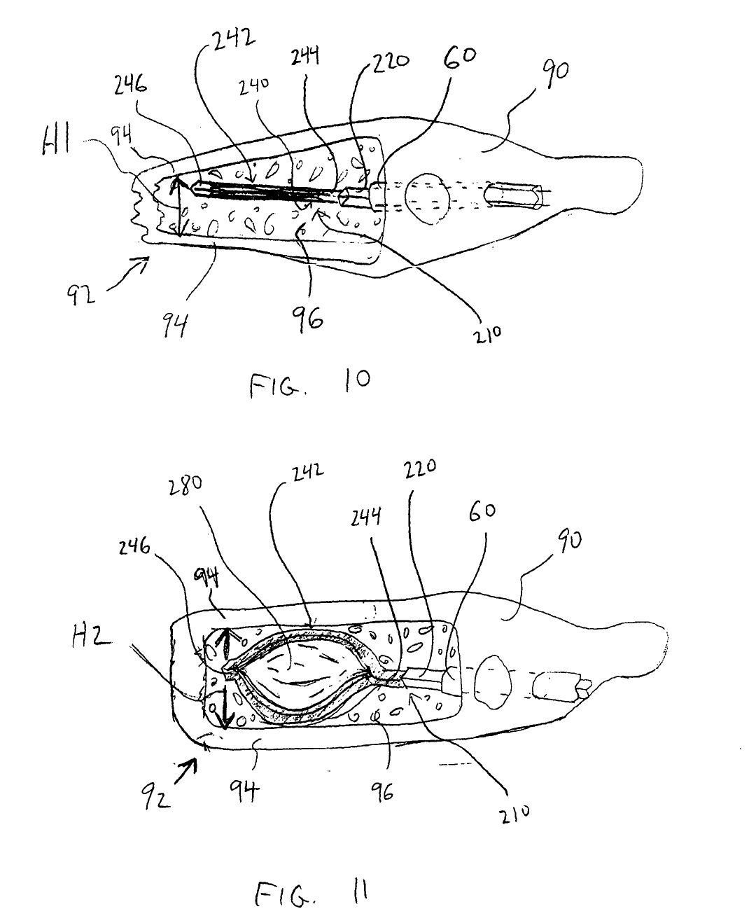

In the embodiment shown, once the balloon 280 has been deflated and removed, a material, such as a bone cement, may then be used to fill a cavity provided by the expansion of the expansible portion 242 without concern about a possible chemical interaction with the balloon 280. Such an embodiment may be useful in situations where the tool 210 is used to restore height to a vertebral body (see FIGS. 10-13), and the material from which the balloon 280 is fabricated is chemically incompatible with a bone cement used to fill the cavity. In such a situation, the expansible portion 242 of the tool 210 will maintain height restored to the vertebral body after the balloon 280 has been deflated and removed. Then, the bone cement may then be inserted, either via a bore extending through the inner shaft 240 of the tool 210, or via a separate cannula (such as a contralateral cannula). The tool 210 may then be removed from the treatment site before the bone cement cures.

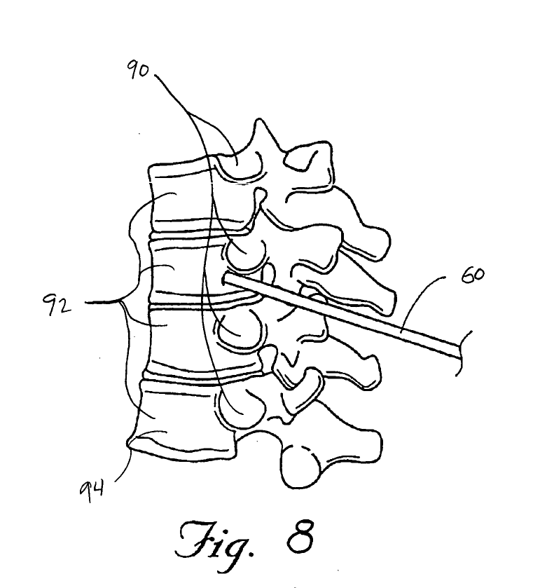

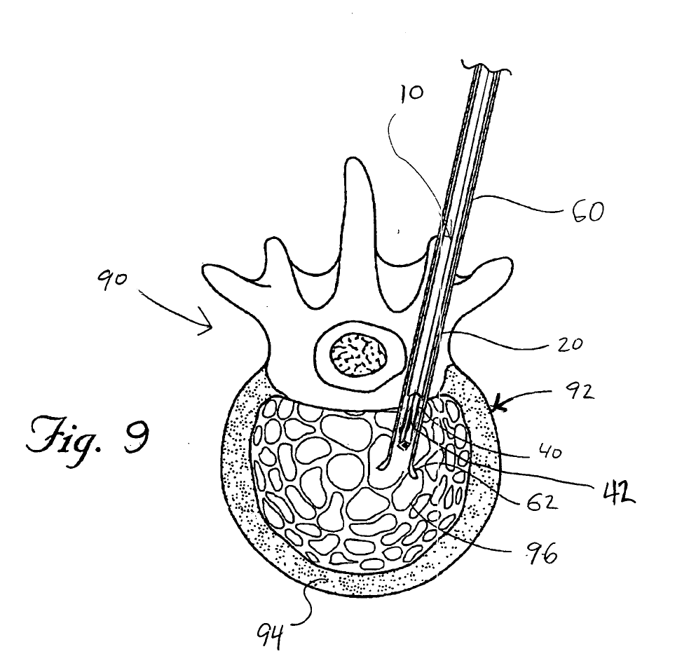

Referring now to FIG. 8, an elevation (lateral) view of several human vertebrae 90 is shown, with a cannula 60 establishing a percutaneous path along its axis to a vertebral body 92 of one of the several vertebrae. The vertebral body 92 extends on the anterior (i.e., front or chest) side of the vertebra 90. The vertebral body 92 comprises an exterior formed from compact cortical bone 94. The cortical bone 94 encloses an interior volume of reticulated cancellous, or spongy, bone 96 (also called medullary bone or trabecular bone—shown in FIGS. 9-12).

The vertebral body 92 is in the shape of an oval disc. As FIGS. 8-12 show, access to the interior volume of the vertebral body 92 can be achieved, e.g., by drilling an access portal through a rear side of the vertebral body 92, (a postero-lateral approach). The portal for the postero-lateral approach enters at a posterior side of the vertebral body 92 and extends anteriorly into the vertebral body 92. The portal can be provided either with a closed, minimally invasive procedure or with an open procedure.

Alternatively, access into the interior volume can be accomplished by drilling an access portal through one or both pedicles of the vertebra 90. This is called a transpedicular approach. It is the physician who ultimately decides which access site is indicated.

A tool according to the present invention may be configured to be deployed adjacent at least one layer of tissue by movement within and along the axis of the cannula 60. For example, as shown in FIG. 8, the cannula 60 may provide a tool, such as the tools 10 or 210 described above, with access to the cancellous bone within the vertebral body 92 of a vertebra 90 to provide a cavity therewithin. Such a cavity may be provided during a procedure for restoring some of the height of a vertebral body lost due to a vertical compression fracture or other pathology or trauma, prior to insertion of a bone cement into the vertebral body 92.

It should be appreciated, however, the systems and methods according to the present invention are not limited in application to human vertebrae, and may be used to provide cavities within or curette other parts of a living or non-living organism. For example, the tool 10 can be deployed in other embodiments in other bone types and within or adjacent other tissue types, such as in a vertebral disc a knee joint, etc.

Referring now to FIG. 9, a plan (coronal) view of a vertebra 90 being accessed by a cannula 60 is shown, with portions removed to reveal cancellous bone 96 within the vertebral body 92. As shown in FIG. 9, the tool 10 as described above with respect to FIGS. 1-6 has been inserted into the cannula 60 for access to the vertebral body 92 of the vertebra 90. The tool 10 is configured to move within and along the axis of the cannula 60.

In the embodiment shown in FIG. 9, the expansible portion 42 of the inner shaft 40 is constrained from expanding to the expanded state shown in FIG. 3 by the inner walls of the cannula 60. However, once beyond a distal end 62 of the cannula 60, the expansible portion 42 may be expanded from the unexpanded state (see FIG. 2) to the expanded state (see FIG. 3), to provide a cavity within the cancellous bone 96.

In use, the outer shaft 20 is carried for sliding and rotation within the cannula 60. The user of the tool 10 may freely slide the outer shaft 20 axially within the cannula 60 to deploy the tool 10 in a targeted treatment site. When deployed at the site, the user can deploy the expansible portion 42 outside the distal end 24 of the outer shaft 20. The user may also able to rotate the outer shaft 20 within the cannula 60 and thereby the expansible portion 42 of the inner shaft 40 to adjust the orientation and travel path of the expansible portion 42.

As shown in FIG. 9, in one embodiment, when fully confined by the cannula 60, the expansible portion 42, even if projecting a significant distance beyond the distal end 24 of the outer shaft 20, is collapsed by the cannula 60. In one such embodiment, the expansible portion 42 of the inner shaft 40 is constrained by the outer shaft 20 if the expansible portion has not been extended beyond the distal end 24 of the outer shaft 20, or by the cannula 60 if extended beyond the distal end 24 of the outer shaft 20 during passage through the cannula 60.

In one embodiment where at least the expansible portion 42 of the inner shaft 40 is fabricated from a shape-memory alloy, once beyond the distal end 62 of the cannula 60, the expansible portion 42 may spring open to assume its preset, native expanded dimension. Thereafter, the physician can operate a controller (such as the controller 48, described above) to alter at least one of the extent to which the expansible portion 42 extends beyond the distal end 62 of the cannula 60, and the extent to which the expansible portion 42 is expanded.

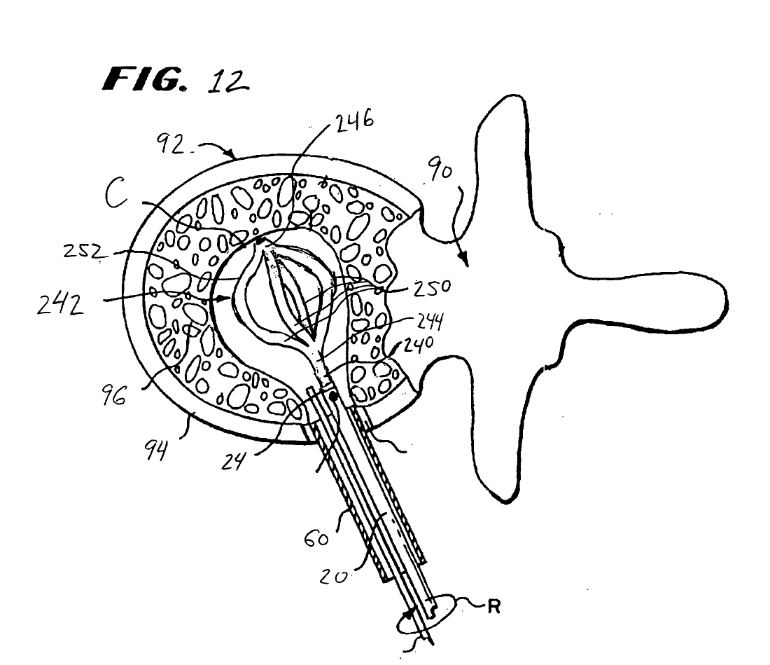

In the embodiment shown in FIG. 9, once the expansible portion 42 is beyond the distal end 62 of the cannula 60, the user of the tool 10 may also rotate the deployed expansible portion 42, by rotating at least one of the outer shaft 20 and the inner shaft 40 within the cannula 60. As discussed below with respect to FIG. 12, rotation of the expansible portion 42 may slice or cut through surrounding tissue mass, in this case, cancellous bone 96.

The materials from which the cannula 60 is fabricated may be selected to facilitate advancement and rotation of the expansible portion 42. The cannula 60 can be constructed, for example, using standard flexible, medical grade plastic materials, such as vinyl, nylon, polyethylenes, ionomer, polyurethane, and polyethylene tetraphthalate (PET). At least some portion of the outer shaft 20 or the inner shaft 40 can also comprise more rigid materials to impart greater stiffness and thereby aid in its manipulation and torque transmission capabilities. More rigid materials that can be used for this purpose comprise stainless steel, nickel-titanium alloys (such as Nitinol), and other metal alloys.

In other embodiments, at least one of the expansible portion 42 and the distal end 62 of the cannula 60 can carry one or more radiological markers, as previously described. The markers may allow radiologic visualization of the expansible portion 42 and its position relative to the cannula 60 while in use within a targeted treatment area.

Referring now to FIG. 10, an elevation (lateral) view of a human vertebra 90 comprising a vertical compression fracture condition is shown. As shown in FIG. 10, a vertebral body 92 of the vertebra 90 has been partially crushed due to an osteoporotic condition of cancellous bone 96 therewithin. The dimension H1 of the vertebral body 92 has been decreased as a result of this fracture. A cannula 60 has been percutaneously inserted to provide access to the cancellous bone 96 within the vertebral body 92.

In the embodiment shown in FIG. 10, the expansible portion 242 of the tool 210 described with respect to FIG. 7 has been inserted into the vertebral body 94 through the cannula 60 in an unexpanded state. The user of the tool 210 may wish to use it to provide a cavity within the vertebral body 92, and to restore height to the vertebral body 92 lost when the fracture occurred.

Referring now to FIG. 11, an elevation (lateral) view of the human vertebra 90 of FIG. 10 is shown after the tool 210 has increased the height of the vertebral body 92 to dimension H2 from dimension H1 as shown in FIG. 10. As shown in FIG. 11, the expansible portion 242 of the inner shaft 240 of the tool 210 has been expanded to a fully expanded state as a result of the inflation of the balloon 280. Both the proximal and distal portions 244, 246, respectively, of the inner shaft 240 have not expanded while the expansible portion 242 has. Such an increase in the dimension H2 may allow a physician using the tool 210 to at least partially restore the vertebra 90 to a shape analogous to its pre-vertical compression fracture condition.

In the embodiment shown in FIGS. 10 and 11, the balloon 280 may be deflated and removed from the tool 210 once the expansible portion 242 has been expanded as shown in FIG. 11. The expansible portion 242 shown is configured to maintain the height restored to dimension H2 after the balloon 280 has been deflated and removed.

In the embodiment shown in FIGS. 10 and 11, the outer shaft 220 and the inner shaft 240 of the tool 210 can be manipulated for axial and rotational movement within the cannula 60. In another embodiment, the outer shaft 220 itself may serve as a cannula. In yet another embodiment, the cannula 60 shown in FIGS. 10 and 11 may act as the outer shaft 220, wherein forces resulting from contact between the expansible portion 242 of the inner shaft 240 and the cannula 60 may cause the expansible portion 242 to contract from the expanded state shown in FIG. 11 to the unexpanded state shown in FIG. 10, or to an intermediate state.

In the embodiment shown in FIGS. 10 and 11, a user of the tool 210 may freely slide at least one of the outer shaft 220 and the inner shaft 240 axially within the cannula 60 to deploy it in the targeted treatment area within the vertebral body 92.

Referring now to FIG. 12, a plan (coronal) view of the tool 210 and vertebra 90 shown in FIGS. 10 and 11 is shown, wherein the tool 210 is shown being rotated in the cancellous bone 96 of the vertebral body 92. As shown in FIG. 12, when the expansible portion 242 of the inner shaft 240 has been expanded, and the balloon 280 (not shown in FIG. 12) has been deflated and removed, the tool 210 may be rotated (as indicated by arrow R) in the cannula 60. In one such embodiment, once expanded, beveled edges 252 on the expansible beams 250 of the expansible portion 242 may act as a curette when the tool 210 is rotated by a user through surrounding cancellous bone 96 (as indicated by arrow R in FIG. 12). Accordingly, the rotating expansible portion 42 may cut cancellous bone 96 in the vertebral body 92. In the embodiment shown in FIG. 12, the beveled edges 252 of the beams 250 are configured to directly contact and shear the cancellous bone 96 to help a user of the tool 210 to provide a cavity C of a desired shape and dimension.

In one embodiment, a suction tube may also be deployed through the cannula 60 to remove cancellous bone cut by the expansible portion 242. In yet another embodiment, at least one of the outer shaft 220 and the inner shaft 240 can comprise an interior lumen to serve as a suction tube as well as to convey a rinsing liquid into the cavity as it is being formed. The suction tube (or a lumen in the outer or inner shafts 220, 240) may introduce a rinsing fluid (with an anticoagulant, if desired) and may remove cancellous bone cut by the expansible portion 242. Alternatively, at least one of the outer shaft 220 and the inner shaft 240 may comprise a first interior lumen that serves as a suction tube, and a second interior lumen that serves to flush the treatment area. In one embodiment, by periodically expanding the expansible portion 242 or rotating the expansible portion 242 once it has been expanded, a user may provide a cavity C having the desired dimensions.

Once the desired cavity C is formed, the cavity-providing tool, such as tool 10 or tool 210, may be withdrawn through the cannula 60. For example, in one embodiment, the user of the tool 210 may first withdraw the expansible portion 242 of the inner shaft 240 into the outer shaft 220, thereby collapsing the expansible portion. The tool 210 may then be removed from a treatment site through the cannula 60. In another embodiment, the expansible portion 242 may be collapsed by simply withdrawing the tool 210 from the treatment site through the cannula 60 without first withdrawing the expansible body 242 into the outer shaft 220.

Alternatively, in one embodiment, the cavity C may be at least partially filled with a material, such as a bone cement, while the expansible portion 242 of the tool 210 is still deployed in an expanded state inside the vertebral body 96. Any other suitable tool can then be deployed through the cannula 60, or through another cannula (such as a contralateral cannula) into the formed cavity C. A second tool can, for example, perform a diagnostic or therapeutic procedure (such as filling the cavity C with a bone cement). In other embodiments other materials (such as a therapeutic material) may be provided into the cavity C by the expansible portion 242 while it is deployed in the vertebral body 92 in the expanded state. For example, an allograft material, a synthetic bone substitute, a medication, or a flowable material that may set to a hardened condition may be provided into the cavity C. The procedure may also be used to apply radiation therapy or chemotherapy. Further details of the injection of such materials 106 into the cavity C for therapeutic purposes may be found in U.S. Pat. Nos. 4,969,888 and 5,108,404 and in co-pending U.S. patent application Publication No. 2003/0229372, which are incorporated herein by reference.

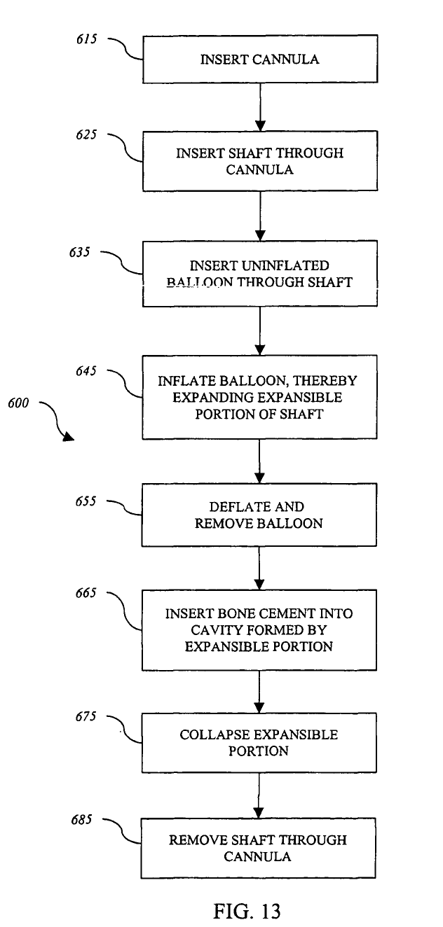

Referring now to FIG. 13, a flow chart of a method 600 according to one embodiment of the present invention is shown. The illustrative embodiment comprises percutaneously inserting a cannula (such as the cannula 60 described above) into a vertebral body of a vertebra comprising a vertical compression fracture condition as shown in box 615.

The method 600 further comprises inserting a shaft comprising an expansible portion through the cannula into the vertebral body as shown in box 625. The expansible portion may comprise, for example, the expansible portion 42 described above, and the shaft may comprise at least the inner shaft 40 described above. In one embodiment the shaft may comprise both the inner and outer shafts 40, 20 described above.

The method 600 further comprises inserting an uninflated balloon (such as the balloon 280 described above) through the shaft to a point within the expansible portion of the shaft as shown in box 635.

The balloon may then be inflated, thereby expanding the expansible portion of the shaft as shown in box 645. The expansion of the expansible portion may provide a cavity (such as the cavity C described above) within the vertebral body.

The method 600 further comprises deflating and removing the balloon, as shown in box 655. For example, the balloon may be deflated and then removed through a bore extending through the shaft while the expansible portion maintains the cavity in the vertebral body.

The method 600 further comprises inserting a bone cement into the cavity formed by the expansible portion while the expansible portion is expanded, as shown in box 665. The bone cement may be inserted through the same cannula that the shaft was inserted through, or may be provided through another cannula into the vertebral body, such as a contralateral cannula. Additionally or alternatively, another surgical tool, such as a scope, may be inserted into the cavity through a bore through the shaft.

The method 600 further comprises collapsing the expansible portion once the bone cement has been inserted into the cavity, as shown in box 675. The expansible portion may be collapsed in one embodiment by forcing the expansible portion to move axially to a point within a distal end of the cannula comprising an inner diameter of lesser dimension than an expanded dimension of the expansible portion. The bone cement, which remains in the cavity, may provide dimensional stability to the vertebral body after the expansible portion of the shaft is collapsed.

The illustrative method 600 finally comprises removing the shaft comprising the expansible portion through the cannula, as shown in box 685. In other embodiments, the expansible portion may be separable from the shaft, and may be left in either an expanded or unexpanded state within the vertebral body while at least some other portion of the shaft is removed through the cannula.

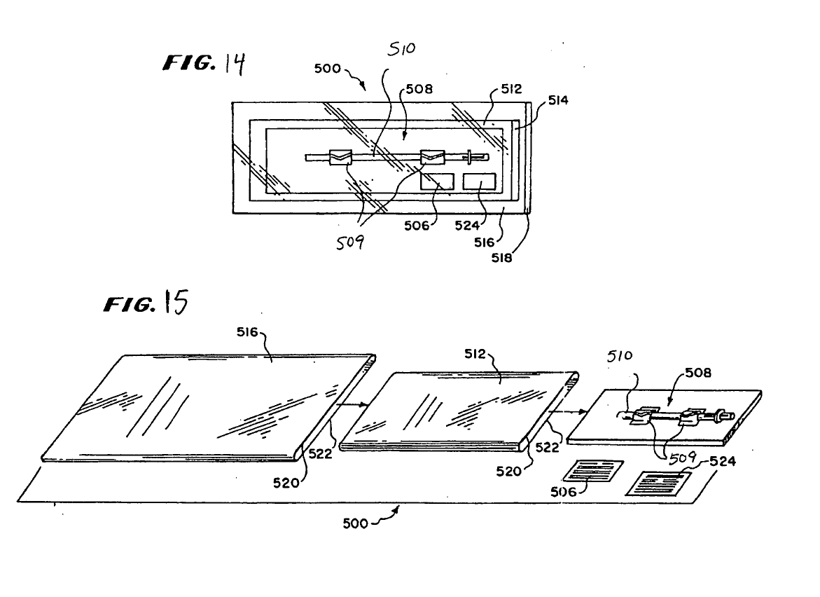

A tool according to one embodiment of the present invention may be packaged in a sterile kit 500 (see FIGS. 14 and 15) prior to deployment in a bone or other tissue. In one such embodiment, the tool may comprise a single use tool.

Referring now to FIGS. 14 and 15, a plan view and an exploded perspective view, respectively, of a sterile kit to store a single use cavity-forming tool according to one embodiment of the present invention is shown. As shown in FIGS. 14 and 15, the kit 500 comprises an interior tray 508. The tray 508 holds the particular cavity-forming tool (generically designated 502) in a lay-flat, straightened condition during sterilization and storage prior to its first use. The tray 508 can be formed from die cut cardboard or thermoformed plastic material. The tray 508 comprises one or more spaced apart tabs 509, which hold the tool 510 in the desired lay-flat, straightened condition.

The kit 500 comprises an inner wrap 512 that, in the embodiment shown, is peripherally sealed by heat or the like, to enclose the tray 508 from contact with the outside environment. One end of the inner wrap 512 comprises a conventional peal-away seal 514 (see FIG. 15), to provide quick access to the tray 508 upon use, which may occur in a sterile environment, such as within an operating room.

The kit 500 shown also comprises an outer wrap 516, which is also peripherally sealed by heat or the like, to enclose the inner wrap 512. One end of the outer wrap 516 comprises a conventional peal-away seal 518 (see FIG. 15), to provide access to the inner wrap 512, which can be removed from the outer wrap 516 in anticipation of imminent use of the tool 510, without compromising sterility of the tool 510 itself.

Both inner and outer wraps 512 and 516 (see FIG. 15) comprise a peripherally sealed top sheet 520 and bottom sheet 522. In the illustrated embodiment, the top sheet 520 is made of transparent plastic film, like polyethylene or MYLAR™ material, to allow visual identification of the contents of the kit 500. The bottom sheet 522 may be made from a material permeable to ethylene oxide sterilization gas, e.g., TYVEC™ plastic material (available from DuPont).

The sterile kit 500 also carries a label or insert 506, which comprises the statement “For Single Patient Use Only” (or comparable language) to affirmatively caution against reuse of the contents of the kit 500. The label 506 also may affirmatively instruct against resterilization of the tool 510. The label 506 also may instruct the physician or user to dispose of the tool 510 and the entire contents of the kit 500 upon use in accordance with applicable biological waste procedures. The presence of the tool 510 packaged in the kit 500 verifies to the physician or user that the tool 510 is sterile and has not been subjected to prior use. The physician or user is thereby assured that the tool 510 meets established performance and sterility specifications, and will have the desired configuration when expanded for use.

The kit 500 also may comprise directions for use 524, which instruct the physician regarding the use of the tool 510 for creating a cavity in cancellous bone in the manners previously described. For example, the directions 524 instruct the physician to deploy and manipulate the tool 510 inside bone to provide a cavity. The directions 524 can also instruct the physician to fill the cavity with a material, e.g., bone cement, allograft material, synthetic bone substitute, a medication, or a flowable material that sets to a hardened condition before, during, or after the tool 510 has provided the cavity.

The foregoing description of the embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

What is claimed is:

1. A system comprising:
a cannula comprising a proximal end, a distal end, an interior bore dimension, and an axis establishing a path; and
a shaft adapted to be deployed adjacent a tissue by movement within and along the axis of the cannula,
the shaft comprising a proximal portion, a distal portion, and an expansible portion disposed between the proximal and distal portions, wherein the expansible portion is constrained from expanding beyond an expanded dimension by the proximal and distal portions.

2. The system of claim 1, wherein the expansible portion is configured to maintain a dimension of a cavity adjacent the tissue while the cavity is filled with a bone cement.

3. The system of claim 1, wherein the expanded dimension is at least the interior bore dimension, and the expansible portion comprises an unexpanded dimension less than the interior bore dimension.

4. The system of claim 3, wherein the expansible portion is configured to be contracted from the expanded dimension to the unexpanded dimension when the expansible portion is brought within the distal end of the cannula from a point beyond the distal end of the cannula.

5. The system of claim 3, wherein the expansible portion is configured to be expanded from the unexpanded dimension to the expanded dimension when the expansible portion is extended to a point beyond the distal end of the cannula.

6. The system of claim 1, wherein the proximal portion of the shaft comprises a cross section substantially similar to a cross section of the distal portion of the shaft.

7. The system of claim 6, wherein the cross sections of the proximal and distal portions both comprise a polygonal shape, and wherein the expansible portion is provided by removing at least one corner of the polygonal shape.

8. The system of claim 1, wherein the expansible portion comprises at least one outwardly expanding beam, wherein the at least one outwardly expanding beam comprises a proximal end coupled to the proximal portion of the shaft, and a distal end coupled to the distal portion of the shaft.

9. The system of claim 1, wherein the expansible portion comprises a surface configured to directly contact and shear the tissue.

10. The system of claim 9, wherein the surface comprises a beveled edge surface.

11. The system of claim 1, wherein the shaft comprises a bore extending therethrough along the axis of the cannula.

12. The system of claim 11, wherein the bore is configured to receive a scope.

13. The system of claim 11, wherein the bore is configured to receive an uninflated balloon.

14. The system of claim 11, wherein the expansible portion is configured to be expanded when the uninflated balloon is inflated.

15. The system of claim 1, wherein at least the expansible portion of the shaft comprises a shape memory material.

16. A method comprising:
inserting a shaft comprising an expansible portion through a cannula, wherein the expansible portion is configured to expand once beyond a distal end of the cannula;
expanding the expansible portion, thereby creating a cavity;
inserting a bone cement into the cavity while the expansible portion is expanded;
collapsing the expansible portion; and
removing the shaft through the cannula.

17. The method of claim 16, wherein expanding the expansible portion comprises:
inserting an uninflated balloon through a bore in the shaft to a point within the expansible portion, wherein the balloon is configured to expand the expansible portion when the balloon is inflated; and
inflating the balloon.

18. The method of claim 17, further comprising:
deflating the balloon; and
removing the balloon through the bore in the shaft.

19. The method of claim 16, wherein the expansible portion comprises a surface configured to directly contact and shear tissue adjacent to the cavity, and further comprising:
contacting the tissue with the surface.

20. The method of claim 19, wherein the surface comprises a beveled edge surface.

21. The method of claim 16, wherein the cannula comprises a first cannula, and wherein inserting the bone cement into the cavity comprises inserting the bone cement through a second cannula.

22. The method of claim 21, wherein the second cannula comprises a contralateral cannula.

23. The method of claim 16, wherein the cavity is adjacent a tissue.

24. The method of claim 16, wherein the expansible portion is disposed between a proximal portion of the shaft and a distal portion of the shaft, and wherein the expansible portion is constrained from expanding beyond an expanded dimension by the proximal and distal portions.

25. The method of claim 24, wherein a cross section of the proximal portion of the shaft is substantially similar to a cross section of the distal portion of the shaft.
